(19) United States
(12) Patent Application Publication
(10) Pub. No.: US 2026/0115328 A1
(43) Pub. Date: Apr. 30, 2026

(54) Adaptable Assessment Platform for Swine

(71) Applicant: The Government of the United States, as Represented by the Secretary of the Army, Fort Detrick, MD (US)

(72) Inventors: Todd Matthew Myers, Middle River, MD (US); Jeffrey Widder, Tabor City, NC (US); Jeffrey Langston, Churchville, MD (US)

(21) Appl. No.: 18/895,513

(22) Filed: Sep. 25, 2024

Related U.S. Application Data
(60) Provisional application No. 63/541,309, filed on Sep. 29, 2023.

(51) Int. Cl. A61K 49/00; A01K 1/03; A01K 5/00; A01K 29/00; A61D 7/00; G01L 5/00

(52) U.S. Cl. CPC A61K 49/0008; A01K 1/031; A01K 5/00; A01K 29/005; A61D 7/00; G01L 5/00

(57) ABSTRACT

An apparatus for assessment of swine includes a housing configured to accommodate swine: at least one response mechanism for recording responses by swine; a light located above and/or below the at least one response mechanism to provide stimuli to the swine; a partition to limit contact with the at least one response mechanism by the swine; and a food delivery and receptacle unit capable of measuring food retrieval by the swine. The apparatus may be used to perform tests on the swine.

This US patent application claims priority to U.S. Ser. No. 63/541,309, filed in the US Patent Office on 29 Sep. 2023, the entirety of which is incorporated herein by reference.

### FIELD OF INVENTION

The present invention is directed to an apparatus and methods for the assessment of swine, for example, minipigs. In particular, the apparatus allows for feeding and testing of swine.

### BACKGROUND OF INVENTION

The minipig (various strains e.g., Gottingen, Yucatan) is fast becoming the standard for assessing dermal chemical hazards because, like most swine, its skin is predictive of human skin response and because this strain's smaller size makes laboratory manipulations and husbandry easier. Unfortunately, standard behavioral tests and apparatus have not been developed for behavioral assessments of swine. Indeed, computer-controlled automated behavioral testing procedures are much needed. The absence of standardized behavioral apparatus and tests may limit or slow adoption of swine (miniature and standard) as an alternative large animal model to dogs and primates.

The inventors successfully developed an apparatus and automated behavioral tests necessary to characterize behavioral function and drug safety/toxicity, setting the stage for subsequent assessments of chemical toxicity and the efficacy of medical countermeasures in swine, for example, minipigs.

### SUMMARY OF INVENTION

The invention provides in a first embodiment an apparatus for the assessment of swine comprising a housing configured to accommodate swine; at least one response mechanism for recording responses by the swine; a light located above and/or below the at least one response mechanism to provide stimuli to the swine; a partition to limit contact by the swine with the at least one response mechanism; and a food delivery and receptacle unit capable of measuring food or liquid retrieval by the swine.

The invention provides in a second embodiment further to any of the previous embodiments an apparatus in which a height of the at least one response mechanism and of the food delivery and receptacle unit may be adjustable.

The invention provides in a third embodiment further to any of the previous embodiments an apparatus in which the at least one response mechanism comprises at least one of a lever, a button, switch, or an omni-directional lever. The at least one response mechanism may comprise at least one, for example a plurality, of upwardly-moveable levers.

The invention provides in a fourth embodiment further to any of the previous embodiments an apparatus in which the partition comprises at least one stationary knob.

The invention provides in a fifth embodiment further to any of the previous embodiments an apparatus in which the food delivery and receptacle unit comprises an adjustable spring-loaded mechanism to measure food retrieval. The adjustable spring-loaded mechanism may activate a microswitch to indicate force applied by swine during food retrieval.

The invention provides in a first method embodiment a method of performing a test of swine comprising feeding swine with the apparatus according to any of the previous embodiments and conducting at least one test on the swine, wherein the at least one test is at least one of a behavioral, cognitive, toxicity, exercise, environmental enrichment, preference testing, or health assessment test.

The invention provides in a second method embodiment further to any of the previous method embodiments a method in which the at least one test comprises a delayed match-to-sample test or a temporal response differentiation test.

The invention provides in a third method embodiment further to any of the previous method embodiments a method in which the at least one test comprises an injury or wounding test.

The invention provides in a fourth method embodiment further to any of the previous method embodiments a method further comprising administering a dose of a drug or chemical agent to the swine. The drug or chemical agent may comprise scopolamine, a muscarinic antagonist, a nerve agent or chemical warfare agent.

The invention provides in a fifth embodiment further to any of the previous method embodiments a method further comprising administering a dose of a drug or chemical agent to the swine, in which the drug or chemical agent comprises a pharmaceutical compound, and the method further comprising assessing the safety or toxicity of the drug or chemical agent.

### BRIEF DESCRIPTION OF THE DRAWINGS

FIG. 1 shows an apparatus for the assessment of swine according to an embodiment of the present invention, with a front view of the at least one response assembly.

FIG. 2 shows a perspective view of the at least one response assembly.

FIG. 3 shows a perspective view of the food delivery (pellet dispenser) and receptacle (food cup) unit.

FIG. 4 is a graph showing percent control response durations under a temporal response differentiation (TRD) schedule of reinforcement as a function of scopolamine dose. Points are mean±SEM. Abscissa is log scaled.

FIG. 5 is a graphs showing percent control response efficiency under a TRD schedule of reinforcement as a function of scopolamine dose. Points are mean±SEM. Abscissa is log scaled.

FIG. 6 shows individual animal response duration distributions under a TRD schedule of reinforcement as a function of scopolamine dose. Gray bars show non-reinforced response durations, dark bars show reinforced response durations. Total responses shown in upper right corner of each figure. Each bin width is 0.1 sec.

FIGS. 7A-7E are graphs showing delayed match-to-sample (DMTS) performance as a function of scopolamine dose. FIG. 7A shows sample stimulus latency; FIG. 7B shows sample stimulus duration; FIG. 7C shows sample stimulus response rate; FIG. 7D shows choice latency as a function of delay; and FIG. 7E shows accuracy as a function of delay.

### DETAILED DESCRIPTION OF INVENTION

The present invention is directed to an apparatus and methods for the assessment of swine. In particular, the apparatus allows for automated behavioral tests of swine. The swine may include, but is not limited to, at least one of Göttingen, Yucatan, Sinclair, Hanford or other small swine strain.

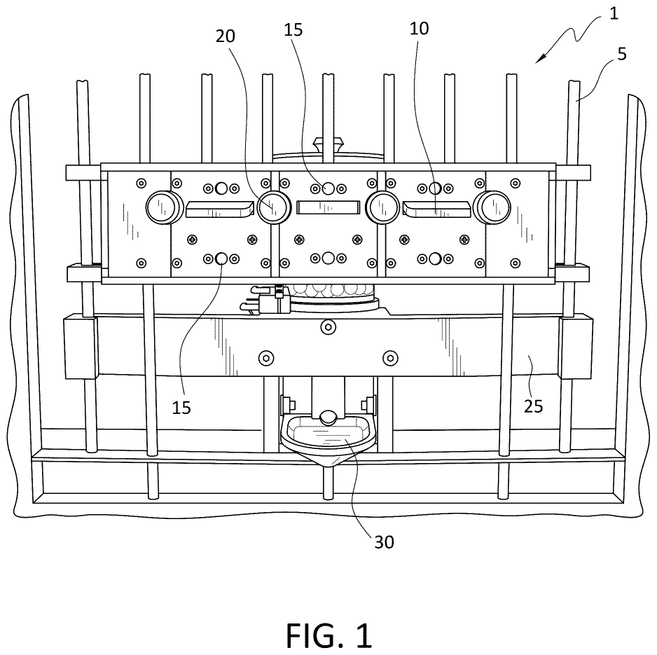

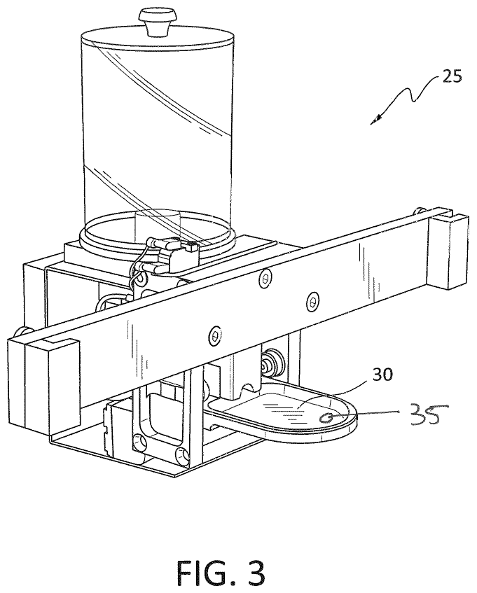

As shown in FIG. 1, an apparatus 1 for the assessment of swine comprises a housing. The housing may be a cage or enclosure configured to accommodate swine of various sizes (e.g. ages) and may have a frame 5. The apparatus includes at least one response mechanism 10 for recording responses by the swine; a light 15 located above and/or below the at least one response mechanism to provide stimuli to the swine; at least one partition 20 to limit contact by the swine with the at least one response mechanism 10; and a food delivery and receptacle unit 25 (FIG. 3) capable of measuring food retrieval by the swine.

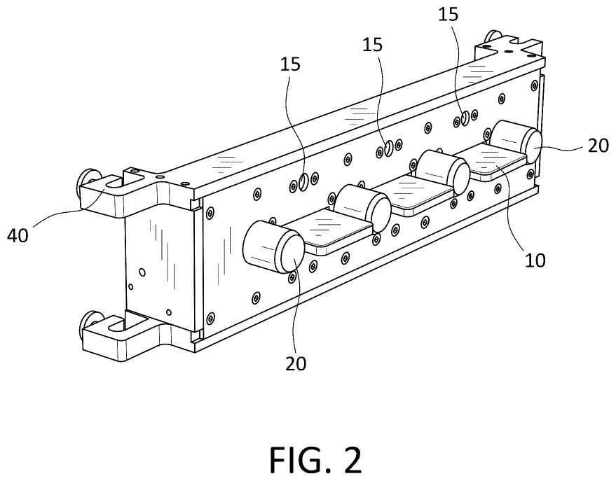

The at least one response mechanism 10 may be at least one of a lever, a button, switch or an omni-directional lever. In specific embodiments, the at least one response mechanism comprises at least one upwardly-moveable lever, for example, a plurality of upwardly-moveable levers (e.g., three or more levers, as shown in FIGS. 1-2). In specific embodiments, the at least one response mechanism may function by a switch closure (e.g., mechanical microswitch) or by photocell beam interruption, magnetic “Reed” switch closure, capacitive contact, or RFID proximity response.

In embodiments, the light 15 may be at least one LED. The light may be recessed.

In embodiments, as shown in FIGS. 1-2, the at least one partition 20 may be at least one stationary knob. The at least one partition may partially limit the swine from activating more than one response mechanism simultaneously.

As shown in FIG. 3, the food delivery and receptacle unit 25 may include a receptacle 30 having a width and depth configured to allow pellet retrieval by the swine. The food delivery and receptacle unit may have a saliva drain hole 35 (FIG. 1), which may be added at the lowest point of the receptacle unit. In specific embodiments, the food delivery and receptacle unit may have an adjustable spring-loaded mechanism to measure food retrieval. The adjustable spring-loaded mechanism may activate a microswitch to indicate force applied by the swine.

In embodiments, the food delivery and receptacle unit may be an assembly capable of dispensing and/or recording pellet retrieval by swine. In a specific embodiment, the food delivery and receptacle unit allows for the automated or scheduled feeding of swine. In embodiments, the food delivery and receptacle unit may provide an auditory signal to alert swine that food has been delivered to the receptacle unit and/or if activated by swine.

In embodiments, the apparatus may comprise 1) a food pellet and receptacle assembly and 2) a response assembly comprising the at least one response mechanism and a light located above and/or below the at least one response mechanism. The two assemblies may be separate and independent modules and therefore may be independently attachable or mountable to the housing and/or frame. The apparatus may also have means, for example ear bars 40 with optional thumbscrews; hooks; clamps; slots; tongue-and-groove (FIG. 2) for adjusting a height of the response assembly and/or of the food pellet delivery and receptacle assembly. In embodiments, thumbscrews may be captive thumbscrew to prevent them from getting separated from the assembly and lost.

According to the present invention, the apparatus may be used to feed swine and conduct at least one test on the swine. The food may include, but is not limited to, pellets or liquids (e.g., consumable beverages such a juice, milk, chocolate milk, smoothies, sugar water, saccharin water, vitamin water, liquid diet such as Ensure, or other consumable liquids or slurries).

The at least one test may be at least one of a behavioral, cognitive, toxicity, exercise, environmental enrichment, preference testing, or health assessment test. In specific embodiments, the at least one test may comprise a delayed match-to-sample (DMTS) test, a temporal response differentiation (TRD) test, or a test for traumatic brain injury, stroke, or Alzheimer's disease.

In other embodiments, the at least one test may comprise an injury or wounding test. The at least one test may include administering a dose of a drug or chemical agent to the swine including, but not limited to, scopolamine, a muscarinic antagonist, a nerve agent or chemical warfare agent, a pharmaceutical compound, or any potential toxicant or chemical of interest. The safety or toxicity of the drug or chemical agent may be assessed. The assessment platform may also be used to study dietary effects, exercise, social hierarchy, or traumatic brain injury of swine.

Advantages of the apparatus of the present invention may include the ability to conduct substantially or completely automated and objective tests in swine of virtually any size and to accommodate the high growth rate via adjustable height of the components or assemblies.

Another advantage is that the food receptacle unit and at least one response mechanism are robust enough to withstand the force applied by swine, which have strong neck muscles. The at least one response mechanism (e.g., levers) is mounted to take advantage of the natural “rooting” or “foraging” behavior of swine, that is a vertical “upward” movement of the snout (as opposed to traditional primate or rat levers that require a downward excursion) to close a microswitch and record a response.

The at least one partition (e.g., stationary knobs) help refine the response, reduce the likelihood of responding to multiple response mechanisms at once, and prevent scratching on the sides of the response mechanisms.

The use of the at least one light and the at least one response mechanism overcomes the disadvantages of touchscreens, as the swine salivate extensively during food-motivated tests, which could obscure the stimuli and record false responses on a touchscreen. As noted, the food receptacle unit may be calibrated with a spring to record pellet retrieval yet be strong enough not to measure incidental contact or become damaged.

In specific embodiments, the inventors utilized VisualBASIC (Microsoft Corporation, Redmond, WA) and a dynamic link library call to actuate relays on an Ontrak USB interface to turn lights off and on, record lever and trough microswitch closures, and deliver pellets, but other software could be used.

In embodiments, the apparatus may have an onboard computer or analyzer that can provide data analysis or control features. For example, the apparatus may have at least one of a Raspberry Pi, Arduino, printed circuit board (PCB), Bluetooth transmitter or other means to control, store, and/or transmit data. This configuration may allow the apparatus to be standalone and not require an external or laptop computer.

### Exemplary Behavioral Tests

Two, non-limiting behavioral tests were selected and adapted from commonly used non-human primate behavioral assessments: a delayed match-to-sample test (a memory test) and a temporal response differentiation test (a time-estimation test). The two tests routinely were included in the Food and Drug Administration's Operant Test Battery with rodents, non-human primates, and humans. These same tests have also been shown to be valuable for assessing nerve agent toxicity and the safety and efficacy of chemical countermeasures. Minipigs were capable of learning both tests and attaining stable performance.

Scopolamine is a muscarinic anticholinergic agent clinically used to reduce nausea and vomiting associated with motion sickness and surgical anesthesia. However, its use in psychological and pharmacological research is as a prototypical amnestic agent in animal models of Alzheimer's disease. Previous research has demonstrated that scopolamine impairs the performance of behavioral memory tests in a variety of animal models. Scopolamine and other muscarinic antagonists such as atropine (broadly described as “anticholinergics”) have also been demonstrated to disrupt operant timing performances. Therefore, after establishing stable baseline performances on both tests, scopolamine dose-response functions were determined to validate the sensitivity of these behavioral tests and to gauge and compare the resulting behavioral perturbations in swine.

Scopolamine dose-effect functions were comparable to those observed in other species, including non-human primates, wherein 37.5 μg/kg of scopolamine (administered intramuscularly) reduced responding approximately 50%.

Thus, the inventors developed an apparatus and automated operant behavioral tests necessary to characterize drug safety in swine. This capability may be valuable, for example, in characterizing chemical agent or drug toxicity, as well as the safety and efficacy of medical countermeasures.

### EXAMPLES

The following are exemplary non-limiting examples of the apparatus and methods of the present invention.

Seven male Göttingen minipigs were obtained from Marshall BioResources (North Rose, NY, USA). Upon arrival, the minipigs were between 36-42 days old and weighed between 2.7-4.1 kg. The animals were acclimated to the facility and observed for evidence of disease for 5 days prior to initiating the study. The colony room was maintained under a 12-h light/dark cycle (lights on at 0600) with temperature (21±2° C.) and humidity (50±10%) controlled. Animals were housed in cages measuring 0.91 m W by 1.22 m L (Lab Products, Seaford, DE). Multiple cages were grouped together by removing interior partitions to afford frequent social housing opportunities.

During feedings and behavioral testing, the central partitions were reinserted to constrain interaction. Animals were fed twice daily and given a small amount of fruit or vegetable enrichment (e.g., string beans, kale) at each afternoon feeding. The total daily caloric allotment equaled 90% of the vendor/breeder recommended amount throughout the study. Water was available ad libitum. Once behavioral training began, the morning rations were reduced to account for the caloric intake earned through training and afternoon rations were adjusted accordingly.

The experimental protocol was approved by the Animal Care and Use Committee at the United States Army Medical Research Institute of Chemical Defense (USAMRICD) and all procedures were conducted in accordance with the principles stated in the Guide for the Care and Use of Laboratory Animals and the Animal Welfare Act of 1966 (P.L. 89-544), as amended.

Behavioral training and testing sessions were conducted in the animal's home cage using a custom designed and fabricated modular home-cage behavioral test panel. The test panel consisted of three components: a food pellet dispenser assembly, a response lever assembly, and a control assembly. The control assembly comprised a personal computer (Dell, Round Rock, TX) and two digital I/O devices (Ontrak Control Systems, Sudbury, Ontario). However, in other embodiments, different I/O components, different control components, or any combination thereof could be used, including a printed circuit board that obviates the laptop and commercial I/O (Ontrak) products.

The food pellet dispenser (MED Associates, Inc., St Albans, VT) was mounted on an aluminum bar (13 mm×50 mm×436 mm [D×H×W]) that attached to the exterior of the animal's cage door with dual thumbscrews at each end. The pellet dispenser was outside of the cage door and an aluminum chute directed food pellets into a food cup (84 mm×70 mm×23 mm [L×W×D]) located on the interior of the cage door through the void typically occupied by the feed trough. The food cup was mounted on a vertically sliding panel maintained under spring tension. Retrieval of the food pellet resulted in activation of a microswitch to monitor pellet retrieval latencies. The food reinforcer was a calorically complete 1000 mg banana or berry flavored Supreme Mini-Treats™

The response lever assembly consisted of three identical response levers (e.g., MED Associates, Inc.) with one tricolor (red, green, and blue; 10 mm D) light-emitting diode (LED) mounted 10 mm above (centered horizontally) the lever and an identical LED mounted 35 mm below (centered horizontally) the lever. The inventors also developed their own aluminum levers that are thicker and more robust than the MED levers. The LEDs for each lever were wired in a parallel circuit. The levers were inverted (requiring upward excursion to register a response) and centered vertically within an aluminum frame (355×31×95 mm [L×D×H]) that was secured to the cage door with thumbscrews located at each corner. Four smooth, round aluminum knobs (25 mm D×25 mm L) were mounted between each lever and at both ends. These stationary knobs were used to prevent animals from contacting multiple levers simultaneously and to encourage contact with the center and front of the lever (rather than the sides). The lever assembly was mounted on the interior of the cage door approximately 25 cm above the floor and adjusted for growth and individual differences to be at eye level for each animal. The computer and digital I/O devices were mounted on an aluminum tray (303 mm×3 mm×449 mm [D×H×W]) that was secured to the upper exterior of the cage door with two thumbscrews, well out of reach of the animals and suitable for human observation and input via the laptop computer. Power and communication cables connected the digital I/O assembly to the levers and pellet dispenser separately on the outside of the cage door.

(−)-Scopolamine hydrobromide trihydrate (hereinafter referred to simply as scopolamine) was obtained from Sigma-Aldrich Corporation (St. Louis, MO; USP product S0929) and dissolved in physiological saline (0.9% sodium chloride in water; Hospira, Lake Forest, IL).

Behavioral testing was conducted with each individual minipig unrestrained in its home cage. Behavioral training and testing sessions occurred at approximately 1030 in the morning five days per week (Monday-Friday). All three components of the modular behavioral test panel were mounted to the cage door at this time and promptly removed and cleaned once the session was completed.

Two animals (Pigs 19-20) initially received one session of autoshaping. However, because lever responding occurred so quickly and spontaneously, the remaining five animals received their initial training session under a fixed-ratio 1 (FR 1) schedule of reinforcement. Four animals (Pigs 19-22) were subsequently trained to perform under a temporal response differentiation (TRD) schedule of food reinforcement. The remaining three animals were trained to perform a delayed match-to-sample (DMTS) task.

### Temporal Response Differentiation (TRD)

A discrete-trials procedure was used and trials were separated by a fixed inter-trial interval (ITI) of 3s, during which all panel LEDs were extinguished and responses were recorded but had no programmed consequences. Each trial began with the illumination of the center lever LEDs green. Upward excursion of the center lever with sufficient force to close the microswitch initiated the response and release of the lever terminated the response. For three animals, food delivery was contingent upon emitting responses with durations between 4.0 and 5.6 s. For Pig 20, the response duration requirement was 2.5 to 3.5 s. A criterion response produced illumination of the food cup and presentation of white noise for 0.5 s (the conditioned reinforcer), accompanied by delivery of one food pellet. The ITI was then initiated. Response durations outside the criterion range extinguished the center lever LEDs and initiated the ITI. Each trial had a 180 second limited hold. If the limited hold elapsed with no response, the trial was recorded as an incomplete trial and immediately initiated the ITI. Sessions ended following 100 reinforcer deliveries or 30 minutes. Animals had an average of 95 (range: 90-100) days of TRD training prior to beginning vehicle and drug injections.

### Delayed Match-to-Sample (DMTS)

Trials were separated by a fixed ITI of 10 s, during which all panel LEDs were extinguished. During the ITI, a response to any lever reset the ITI. Each trial began with the presentation of the sample stimulus. The sample stimulus consisted of the illumination of the center lever LEDs with red or blue. Completion of 5 responses on the center lever extinguished the sample stimulus LEDs and initiated the delay interval (0.1-5 s). Center lever responses during the delay interval were recorded but had no programmed consequences. Following the delay interval, choice stimuli were presented. The choice stimuli always presented illuminated red LEDs on one lever and blue LEDs on the other lever, but the location of the blue or red correct choice stimulus was pseudo-randomly controlled such that each color was presented equally often in each location. A response to the side lever with the same color LED as the recently presented sample stimulus resulted in a pellet delivery accompanied by white noise presentation and illumination of the food cup LED for 0.5 s. Conversely, a response to the lever with the non-matching color, extinguished all lever lights and ended the trial. The sample stimulus color and correct choice stimulus position (left or right) were counterbalanced and varied pseudo-randomly with the restriction that an equal number of trials of each color occurred in each position at each delay. Under terminal parameters, the three delays used were typically 0.1, 2, and 5 s, but varied slightly across individuals to produce comparable levels of accuracy at the short, medium, and long delays, respectively. A 30-s limited hold was imposed on the sample-stimulus period and a 15-s limited hold was imposed on the choice period. Sessions ended following the completion of 108 trials or the delivery of 100 reinforcers, whichever occurred first. Animals had an average of 65.75 (range: 65-66) days of training prior to beginning vehicle and drug injections.

### Drug Administration

Once behavioral performances had stabilized, scopolamine doses (18.75, 37.5, and 75 μg/kg; equivalent to 16.44, 32.88, and 65.75 μg/kg scopolamine HBr and 12.98, 25.95, and 51.91 μg/kg scopolamine base, respectively) were administered in a quasi-random order. Injections were administered intramuscularly into the lateral thigh and injection volumes were <0.5 mL. Saline vehicle injections (0.5 mL) were administered on a near-daily basis. Drug injections were separated by at least three days. Injection location (left vs. right leg) was alternated across days. Behavioral sessions began 5 minutes after injection.

### Data Analysis

Scopolamine effects on TRD performance were calculated as a percentage of vehicle-injection control sessions. Scopolamine effects on DMTS performance were compared to vehicle injection control performance at each delay value. Statistical evaluation of scopolamine effects on TRD performance were accomplished using a linear mixed effects model with scopolamine dose as the fixed effect and subject as a random effect. Statistical analysis of scopolamine effects on DMTS were evaluated using a linear mixed effects model with scopolamine dose, delay, and their interaction as fixed effects and subject as the random effect. Significant main effects were followed by pairwise comparisons between all levels of the fixed-effect. Significant interactions were followed by analyses examining simple contrasts to examine the main effects. A significance level of p<0.05 was used for all tests. Adjustments for multiple comparisons were made using Bonferroni's correction procedure.

### Results

### Temporal Response Differentiation

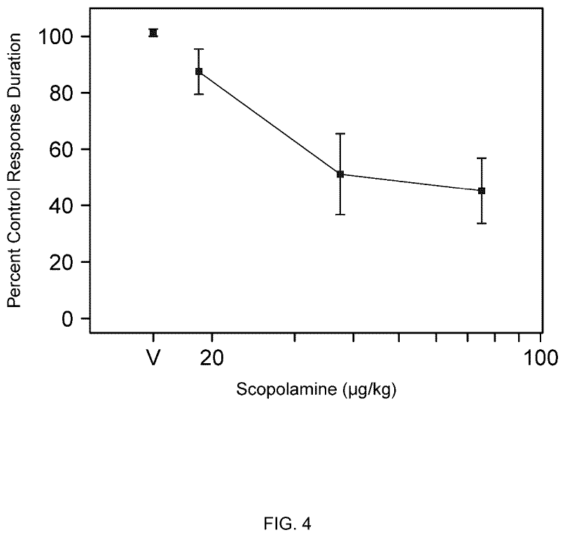

FIG. 4 shows the effects of scopolamine on mean percent vehicle control response durations. As shown, mean response durations decreased as a function of scopolamine dose. Statistical analysis of response durations indicated a significant main effect of scopolamine dose F(3.46)=38.02, p<0.0001. Pairwise comparisons between doses indicated that scopolamine at 18.75 μg/kg decreased mean response durations by approximately 13% which was not statistically different than under vehicle control conditions (p>0.29). However, mean percent vehicle control response durations following scopolamine at doses of 37.5 and 75 μg/kg were significantly decreased compared to both vehicle control and those following scopolamine at 18.75 μg/kg (p<. 0.002).

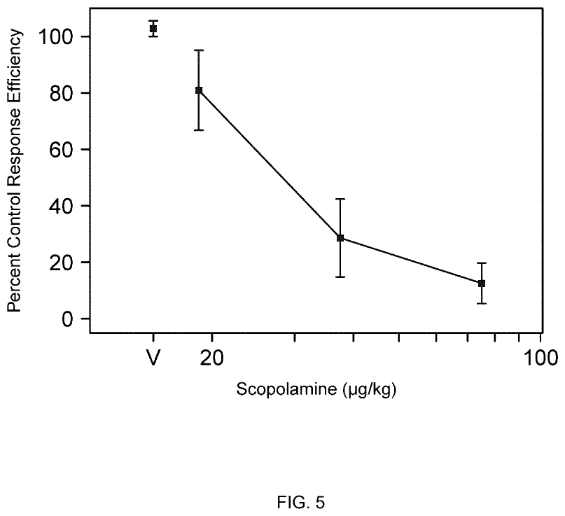

FIG. 5 shows percent vehicle control response efficiency (total reinforcers to total responses). As shown, response efficiency decreased as a function of scopolamine dose. Statistical analyses revealed a significant main effect of scopolamine dose on response efficiency F(3.46)=39.02, p<0.0001. Pairwise comparison between doses indicated that response efficiency following 18.75 μg/kg of scopolamine decreased by approximately 20%, which was not significantly different than under vehicle control conditions (p>0.26). However, response efficiency following scopolamine at doses of 37.5 and 75 μg/kg decreased to 28% (p<0.0001) and 12.5% (p<0.0001) of vehicle control, respectively. Response efficiency following these two doses (37.5 and 75 μg/kg) were not significantly different from each other; however, response efficiency following these two doses were significantly different than that following 18.75 μg/kg scopolamine (p<0.003).

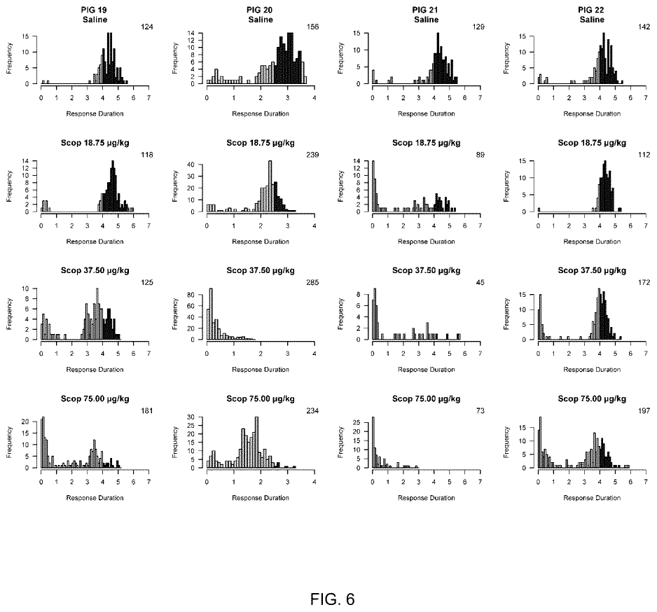

FIG. 6 shows response duration distributions for individual animals (Pigs 19-22) under vehicle control conditions and following each dose of scopolamine. Each animal's data are arranged in a single column with vehicle control in the first row and increasing scopolamine doses in subsequent rows. Response duration distributions under the TRD schedule following saline administration tended to have a single mode within the reinforced band. With increasing doses of scopolamine, the response duration distributions tended to flatten and shift leftward. At the two highest doses of scopolamine (37.5 and 75 μg/kg) response distributions were shifted leftward toward shorter response durations and a second mode appeared at very short response durations (<0.3 s).

### Delayed Match-to-Sample

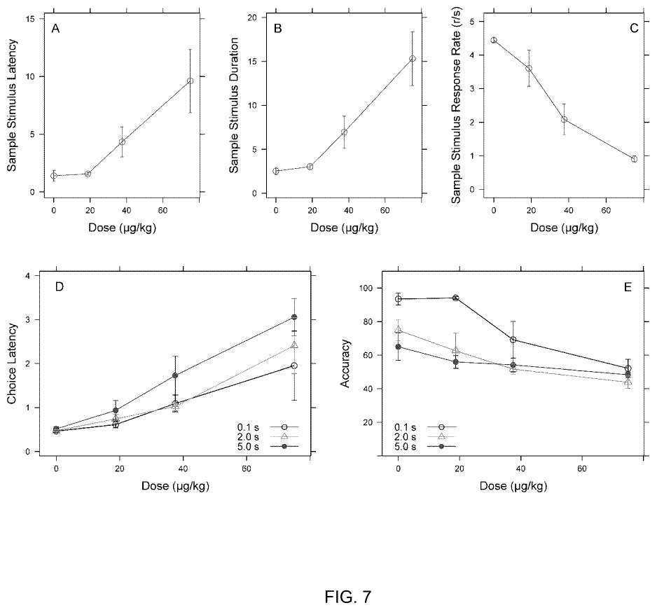

FIGS. 7A-7E shows the effects of scopolamine dose on DMTS performance. FIG. 7A shows the effects of scopolamine dose on sample stimulus latency. Statistical analysis indicated a significant effect of scopolamine dose on sample stimulus latency F(3.47)=4.87, p<0.005. Pairwise comparison indicated that sample stimulus latency following 75 μg/kg scopolamine was significantly delayed compared to vehicle control (p<0.004).

FIG. 7B shows sample stimulus duration as a function of scopolamine dose. Statistical analysis indicated a significant main effect of scopolamine dose F(3.47)=11.32, p<0.001. Pairwise comparisons revealed that sample stimulus duration following the highest dose of scopolamine (75 μg/kg) was significantly longer than that following vehicle (p<0.0001) and 18.75 μg/kg scopolamine (p<0.002).

FIG. 7C shows sample stimulus response rate as a function of scopolamine dose. As seen in this panel, sample stimulus response rate decreased as a function of increasing scopolamine dose. Statistical analysis indicated a significant main effect of scopolamine dose on response rate F(3.47)=35.36, p<0.001. Pairwise comparisons revealed no significant differences in sample stimulus response rates following vehicle or 18.75 μg/kg scopolamine. Furthermore, there were no significant differences in sample stimulus response rates following the two highest doses of scopolamine (37.5 and 75 μg/kg). However, sample stimulus response rates following the two highest doses of scopolamine were significantly different than those following both vehicle and the lowest dose of scopolamine (18.75 μg/kg), p<0.05.

FIG. 7D shows the effects of scopolamine on choice latency as a function of the delay interval. As seen in this panel, choice latency increased as a function of scopolamine dose for the three delay intervals. Statistical analysis indicated a significant main effect of dose F(3.145)=27.08, p<0001 and a significant interaction between dose and delay interval F(6.145)=4.28, p<0005. The main effect of delay was not significant F(2.145)=0.41, p>0.66. Pairwise comparisons of choice latency between scopolamine doses averaged over all delay values revealed a significant dose-response effect (vehicle<18.75 μg/kg<37.5 μg/kg<75 μg/kg; p<04 for all; see Table 1). Tests of simple main effects indicated that choice latencies at the shortest (0.1 s) and intermediate (2.0 s) delays following 75 μg/kg scopolamine were significantly greater than those following vehicle and the two lower doses of scopolamine (18.75 and 37.5 μg/kg; p<0.003). Furthermore choice latencies at the 0.1 s and 2.0 s delays following 37.5 μg/kg were significantly greater than those following vehicle. At the longest delay (5.0 s), choice latencies following 18.75 μg/kg scopolamine were not significantly different that those following vehicle. However, choice latencies at the 5.0 s delay following both 37.5 and 75 μg/kg scopolamine were significantly longer than those following vehicle (p<0.0001). Additionally, at the longest delay, choice latency significantly increased with scopolamine dose (18.75 μg/kg<37.5 μg/kg<75 μg/kg; p<0.01).

TABLE 1
Mean choice latency (95% CI) as a function
of Scopolamine Dose and Delay value.

| | | Delay Interval | | | |
|---|---|---|---|---|---|
| Scopolamine | | 0.1 s | 2 s | 5 s | Average |
| 0.00 | μg/kg | 0.46 | 0.47 | 0.52 | 0.48 |
| | | (0.42-0.50) | (0.45-0.49) | (0.43-0.60) | (0.44-0.52) |
| 18.75 | μg/kg | 0.61 | 0.74 | 0.93 | *0.76 |
| | | (0.29-0.93) | (0.30-1.18) | (−0.02-1.89) | (0.27-1.25) |
| 37.50 | μg/kg | *1.09 | *1.02 | *^{†}1.73 | *^{†}1.28 |
| | | (0.27-2.16) | (0.59-1.46) | (−0.17-3.63) | (0.40-2.16) |
| 75.00 | μg/kg | *^{†‡}1.95 | *^{†‡}2.41 | *^{†‡}3.05 | *^{†‡}2.47 |
| | | (−1.44-5.35) | (−0.32-5.14) | (1.22-4.89) | (0.28-4.66) |

*Significantly different from vehicle (0.00 μg/kg);
^{†}Significantly different from 18.75 μg/kg scopolamine.
^{‡}Significantly different from 37.50 μg/kg scopolamine.

FIG. 7E shows the effects of scopolamine dose on DMTS accuracy as a function of delay interval. Statistical analyses indicated a significant main effect of scopolamine dose F(3,145)=17.73, p<0.0001, a significant main effect of delay interval F(2, 145)=79.74, p<0.0001, and a non-significant interaction between dose and delay interval F(6, 145)=2.06, p>0.06. Pairwise comparison of DMTS accuracy as a function of scopolamine dose indicated that accuracy at the two highest doses of scopolamine (37.5 and 75 μg/kg) was significantly lower than that following vehicle (p<0.001). Additionally, accuracy following the highest dose of scopolamine (75 μg/kg) was significantly lower than that following the lowest dose of scopolamine (18.75 μg/kg). Pairwise comparisons of DMTS accuracy as a function of delay interval revealed that accuracy at the intermediate (2.0 s) and longest (5.0 s) delays was significantly lower than that at the shortest (0.1 s; p<0.001) delay; however, accuracy at the two longest delays was not significantly different (p=1.0). Given the marginal significance of the interaction, an analysis of simple contrasts was conducted. The analysis of accuracy at each delay indicated that at the shortest delay (0.1 s) DMTS accuracy following the highest dose of scopolamine (75 μg/kg) was significantly lower than that following the lowest dose of scopolamine (18.75 μg/kg; p<0.0001) and that under vehicle control conditions (p<0.001). Likewise, at the shortest delay (0.1 s) DMTS accuracy following the intermediate dose of scopolamine (37.5 μg/kg) was significantly lower than that under vehicle control conditions (p=0014) and following the lowest dose of scopolamine (18.75 μg/kg; p=0.032). Additionally, at the intermediate delay (2.0 s) DMTS accuracy under vehicle control conditions was significantly higher than that following both the intermediate dose of scopolamine (37.5 μg/kg; p=0.0026) and the highest dose of scopolamine (75 μg/kg; p<0.0001). At the longest delay (5.0 s), there was a marginally statistically significant difference in DMTS accuracy between vehicle control conditions and that following the highest dose of scopolamine (75 μg/kg; p=0.0673).

### DISCUSSION

The inventors successfully demonstrated the ability of male Göttingen minipigs to perform operant tests that are routinely valued for assessing the safety of pharmacological compounds. This work utilized scopolamine and demonstrated a graded dose-effect function with both DMTS and TRD. For TRD, scopolamine produced a marked dose-dependent reduction in response efficiency, observed primarily as a suboptimal leftward shift in the response duration distribution, with an increase in brief and very brief response durations.

Scopolamine effects in minipigs are comparable to those observed in other species under similar schedules of reinforcement. For DMTS, effects of delay and dose interacted for choice latency, with the low scopolamine dose significantly increasing choice latency at only the longest delay, and moderate and high scopolamine doses increasing choice latency comparably at all delays. For accuracy, a more generalized dose-dependent suppression was observed with increasing scopolamine doses. These DMTS findings directly mirror those previously published by several laboratories using Old World monkeys such as rhesus macaques and African green monkeys, as well as humans.

In embodiments, the present invention sets a foundation for establishing standardized operant behavioral tests in this species and for evaluating not only the safety of pharmacological compounds, but also the toxicity and time course of chemical threat agents (applied dermally, ingested, injected, or inhaled) and the efficacy of medical countermeasures regimens on short-term and long-term operant behavioral outcomes.

Although the present invention has been described in terms of particular exemplary and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

What is claimed is:

1. An apparatus for assessment of swine, comprising:
a housing configured to accommodate swine;
at least one response mechanism for recording responses by the swine;
a light located above and/or below the at least one response mechanism to provide stimuli to the swine;
a partition to limit contact by the swine with the at least one response mechanism; and
a food delivery and receptacle unit capable of measuring food or liquid retrieval by the swine.

2. The apparatus of claim 1, wherein a height of the at least one response mechanism and of the food delivery and receptacle unit are adjustable.

3. The apparatus of claim 1, wherein the light is an LED.

4. The apparatus of claim 1, wherein the at least one response mechanism comprises at least one of a lever, switch, a button, or an omni-directional lever.

5. The apparatus of claim 1, wherein the at least one response mechanism comprises at least one upwardly-moveable lever.

6. The apparatus of claim 1, wherein the at least one response mechanism comprises a plurality of upwardly-moveable levers.

7. The apparatus of claim 1, wherein the partition comprises at least one stationary knob.

8. The apparatus of claim 1, wherein the food delivery and receptacle unit comprises a receptacle having a width and depth configured to allow pellet retrieval by the swine.

9. The apparatus of claim 1, wherein the food delivery and receptacle unit comprises a saliva drain hole.

10. The apparatus of claim 1, wherein the food delivery and receptacle unit comprises an adjustable spring-loaded mechanism to measure food retrieval.

11. The apparatus of claim 10, wherein the adjustable spring-loaded mechanism activates a microswitch to indicate force applied by the swine.

12. The apparatus of claim 1, wherein the swine comprise at least one of Göttingen, Yucatan, Sinclair, Hanford or other small swine strain.

13. An apparatus for assessment of swine, comprising:
a food pellet delivery and receptacle assembly capable of recording pellet retrieval by swine; and
a response assembly having at least one response mechanism and a light located above and/or below the at least one response mechanism to provide stimuli to the swine,
wherein each assembly is independently attachable or mountable to a cage or housing for the swine.

14. The apparatus of claim 13, further comprising at least one partition to partially limit the swine from activating more than one response mechanism simultaneously.

15. A method of performing a test of swine, comprising:
feeding swine with the apparatus of claim 1; and
conducting at least one test on the swine, wherein the at least one test is at least one of a behavioral, cognitive, toxicity, exercise, environmental enrichment, preference testing, or health assessment test.

16. The method of claim 15, wherein the at least one test comprises a delayed match-to-sample test or a temporal response differentiation test.

17. The method of claim 15, wherein the at least one test comprises an injury or wounding test.

18. The method of claim 15, further comprising administering a dose of a drug or chemical agent to the swine.

19. The method of claim 18, wherein the drug or chemical agent comprises scopolamine, a muscarinic antagonist, or a nerve agent or chemical warfare agent.

20. The method of claim 18, wherein the drug or chemical agent comprises a pharmaceutical compound and said method further comprising assessing the safety or toxicity of the drug or chemical agent.
